(19) United States
(12) Patent Application Publication
(10) Pub. No.: US 2016/0067167 A1
(43) Pub. Date: Mar. 10, 2016

(54) ORAL CARE COMPOSITIONS FOR TREATMENT OF SENSITIVE TEETH

(71) Applicant: ISP INVESTMENTS INC., Wilmington, DE (US)

(72) Inventors: William E. Prosise, Ramsey, NJ (US); Gillian Giannone, Wayne, NJ (US); Jeffrey Lynn, Long Valley, NJ (US); Cristian Grigoras, Suffern, NY (US); Jenn Shih, Paramus, NJ (US)

(73) Assignee: ISP INVESTMENTS INC., Wilmington, DE (US)

(21) Appl. No.: 14/442,306

(22) PCT Filed: Nov. 15, 2013
(86) PCT No.: PCT/US13/70220

§ 371 Date: May 12, 2015

Related U.S. Application Data
(60) Provisional application No. 61/726,860, filed on Nov. 15, 2012.

(51) Int. Cl. A61K 8/81; A61Q 11/00

(52) U.S. Cl. CPC A61K 8/8164; A61K 8/8129; A61Q 11/00; A61K 2800/594; A61K 2800/92

(57) ABSTRACT

The present invention provides oral care compositions for treating tooth sensitivity. The composition comprises a synergistic mixture containing a copolymer having at least one repeating unit of alkyl vinyl ether functionality and a monomer having at least one repeating unit of ethylenically unsaturated dicarboxylic acid or anhydride functionality present in an amount of about 0.1 to about 50% (w/w) of the total mixture; and a homo polymer having repeating unit of alkyl vinyl ether functionality present in an amount of about 0.1 to about 50% (w/w) of the total mixture. These synergistic mixtures of polymers act as barriers for dentin flow and reduce pain and discomfort caused by tooth sensitivity and exhibits at least 60% average reduction in dentin flow.

### FIELD OF THE INVENTION

The invention relates to oral care compositions for the treatment of dental ailments and more specifically, to methods and compositions that show improved barrier properties to teeth against dentin hypersensitivity or tooth sensitivity.

### BACKGROUND OF THE INVENTION

Tooth sensitivity or dentin hypersensitivity is caused by the movement of fluid within tiny tubes located in the dentin (the layer of tissue found beneath the hard enamel that contains the inner pulp), which results in nerve irritation. When the hard enamel is worn down or gums have receded, tiny tubes on the surface are exposed. Exposed areas of the tooth can cause pain and even affect or change one's eating, drinking, and breathing habits. Pain can be caused by eating hot or cold foods or drinking beverages, touching the teeth, or exposing them to cold air. The excessive consumption of acid-containing foods and beverages, such as citrus juices and fruits and soft drinks, can also cause tooth sensitivity. Bulimia and acid reflux can also result in erosion of the hard enamel and sensitivity due to acid in the mouth.

One approach for the treatment of tooth sensitivity is reducing the excitability of tooth nerve by interfering with the ordinary triggering process of nerve. This is achieved by altering the chemical environment of the nerve.

U.S. Pat. Nos. 3,863,006, 3,888,976, 4,631,185, 4,751,072, 4,990,327 disclose various potassium and strontium salts for reducing the nerve excitation.

U.S. Pat. No. 3,122,483 assigned to Block Drug Company Inc. discloses water containing tooth paste for the treatment of hypersensitive tooth characterized by including therein water-soluble strontium compounds that release strontium ions from solutions.

Another approach to treat sensitive teeth is by blocking the dentinal tubules so that dentin flow is reduced, impulses do not reach the nerve thereby reducing or eliminating the pain and discomfort caused by tooth sensitivity.

U.S. Pat. Nos. 4,634,589, 4,710,372, 4,362,713 and 5,885,551 disclose “tubule blocking agents” such as apatite particles; hydroxyl apatite or fluoroapatite particles, 1-alkene/maleic acid copolymers and salts of alginic acid respectively.

U.S. Pat. No. 5,188,818 assigned to ISP Investments Inc. discloses an oral care composition comprising about 1-20% by weight strontium salt of copolymer of maleic anhydride and methyl vinyl ether.

U.S. Pat. No. 5,270,031 assigned to Block Drug Company Inc. discloses water swellable polyacrylic acid salt having a degree of neutralization of about 20 to 100%. The poly acrylate salts can comprise the anionic, cationic or amphoteric forms of methyl vinyl ether and maleic anhydride copolymers of polyacrylic acid polymers with sodium, calcium, potassium, ammonium, zinc and other metals.

U.S. Pat. No. 4,952,558 assigned to BASF and U.S. Pat. No. 5,202,112 assigned to Colgate disclose branched copolymer of alkylvinyl ether and maleic anhydride. However tooth sensitivity is not covered in these patents.

U.S. Pat. No. 5,032,637 assigned to Adhesives Research Inc. discloses water-inactivatable pressure-sensitive adhesive comprising of a hydrophilic base polymer in admixture with a water-soluble tackifying agent. The said base polymer comprises at least one polymer of an alkyl vinyl ether/maleic acid ester in an admixture with at least one polyvinyl alkyl ether. However oral care application or tooth sensitivity is not disclosed in the patent.

U.S. Pat. No. 6,211,318 assigned to ISP Investments Inc. discloses solvent free fine white powders of a copolymer of maleic anhydride and a C₁-C₄ alkyl vinyl ether which is substantially free of poly(alkyl vinyl ether) homopolymer. The process involves removal of homopolymer whose presence as impurity imparts objectionable color and haze to personal and oral care compositions.

U.S. Pat. No. 6,241,972 assigned to Block Drug Company Inc. discloses compositions and their use in treating dentinal hypersensitivity comprising a copolymer having repeating units of hydrophilic monomer such as a carboxylic acid, a dicarboxylic acid or a dicarboxylic acid anhydride and a hydrophobic monomer consisting of an α-olefin having at least eight carbon atoms, full and partially hydrolyzed forms thereof and full and partial salts thereof. A preferred desensitizing agent is PA-18 which is an alternating copolymer of a 1:1 molar ratio of maleic anhydride and 1-octadecene. Desensitizing agent is selected from the group consisting of tetradecene/maleic anhydride copolymer, octadecene/maleic anhydride copolymer and triacontene/maleic anhydride copolymer. Chevron PA-18 is used in Sensodyne toothpaste.

US Patent Appln No. 201000322984 assigned to GlaxoSmithKline discloses inorganic oxide particles surface coated with an anionic polymer and an orally acceptable carrier or excipient. Anionic polymer is a polycarboxylate preferably octadecene maleic anhydride copolymer.

Despite the solutions proposed by the aforementioned patents and other approaches described in the published literature, there still exists a need for an effective oral care composition to reduce the pain and discomfort arising out of tooth sensitivity.

Thus it is always desired to explore more avenues for better treatment of tooth sensitivity characterized by decrease in dental fluid flow (or dentin permeability) by occlusion of dentin tubules.

The inventive polymeric oral care compositions particularly for treating tooth sensitivity provide all such desired properties of reduction in dentin flow or permeability.

### SUMMARY OF THE INVENTION

We have invented a new, improved and effective oral care composition for treating tooth sensitivity effects. The invention provides compositions and methods for delivering tooth anti-sensitivity effects.

In accordance with the present invention, there is provided an oral care composition for treating tooth sensitivity comprising a synergistic mixture of

component (A)—a copolymer having at least one repeating unit of alkyl vinyl ether functionality and a monomer having at least one repeating unit of ethylenically unsaturated dicarboxylic acid or anhydride functionality present in an amount of about 0.1 to about 50% (w/w) of the total mixture; and
component (B)—a homo polymer having repeating unit of alkyl vinyl ether monomer present in an amount of about 0.1 to about 50% (w/w) of the total mixture;
wherein the composition exhibits at least 60% average reduction in dentin flow.

In one of the preferred embodiments, the repeating unit (A) of alkyl vinyl ether functionality is selected from the group comprising C₁ to C₂₂ alkyl vinyl ethers including methyl vinyl ether, ethyl vinyl ether, iso-propyl vinyl ether, n-propyl vinyl ether, n-butyl vinyl ether, iso-butyl vinyl ether, tert-butyl vinyl ether, 2-ethylhexyl vinyl ether, n-octyl vinyl ether, n-dodecyl vinyl ether, hexadecyl vinyl ether, octadecyl vinyl ether, isopropenyl methyl ether, 1-butyl vinyl ether, hydroxybutyl vinyl ether, 1,4-cyclohexane-dimethanol monovinyl ether, methoxyethyl vinyl ether, methoxyethoxyethyl vinyl ether, or monomethyl ether of triethylene glycol vinyl ether.

The repeating unit of ethylenically unsaturated dicarboxylic acid or anhydride functionality is selected from maleic acid, maleic anhydride, citraconic acid, citraconic anhydride, itaconic acid, itaconic anhydride, ethylmaleic acid, ethylmaleic anhydride, dimethyl maleic acid, dimethyl maleic anhydride, chloromaleic acid or chloromaleic anhydride.

Typically, synergistic mixture of polymers comprise component (A) in an amount of about 0.1 to about 50% (w/w) and component (B) in an amount of about 0.1 to about 50% (w/w) of the total oral care composition.

Typically, synergistic mixture of polymers comprises components (A) and (B) present in weight ratio of A to B from 2 to 1 to about 10 to 1.

The present invention provides oral care compositions comprising synergistic mixture of methyl vinyl ether/maleic anhydride (or maleic acid) copolymer and poly (methyl vinyl ether).

The present invention further provides an oral care composition comprising a synergistic mixture of about 2% w/w of methyl vinyl ether/maleic anhydride (or maleic acid) copolymer; 0.5% of poly (methyl vinyl ether); and about 98% w/w from about of oral care additives.

The polymer content in the oral care composition is preferably from about 0.1% (w/w) to about 10% (w/w) of the total oral care composition.

The synergistic mixture of polymers in the oral care composition has component A with a molecular weight of about 100,000 to 10,000,000 daltons and component B with a molecular weight of about 2,000 to 1,000,000 daltons.

The oral care composition further comprises oral care additives selected from antibacterial agents, anti-inflammatory agents, humectants, surfactants, detergents, anticaries agents, whitening agents, anti-cavity or enamel repair agents, breath freshening agents, sweeteners, flavor oils, foaming agents, abrasives, tartar control or anti-stain ingredients, anti-erosion ingredients, fluoride sources, gums, stabilizers, opacifying agents, pH buffering agents, chelating agents, colorants, enzymes, sensate ingredients, and/or their combinations.

The present invention makes uses of a method of measuring the hydrodynamic flow through human dentin using an industry standard test method. Such measurement of dentin flow can be used as a means to evaluate the tubule occlusion effects of oral care preparations. Active ingredients that provide greater tubule occlusion possess greater potential in reducing the tooth sensitivity.

Further, the present invention provides an oral care composition which shows an average reduction of dentin flow of about 60-95%, preferably 70-95% and more preferably 75-92%.

Typically the inventive compositions can be incorporated into formulations such as toothpastes, powders, sprays, suspensions, varnishes, sealants, coatings, oral strips, mouth washes, mouth rinses, teeth whitening compositions or boosters, anti-calculus formulations, anti-gingivitis formulations, instant powders, pastilles, oral gels, chewing gums, buccal patches, or lozenges.

The inventive compositions are used for alleviating the symptoms of tooth sensitivity, acid erosion, or caries comprising topically applying the oral care composition on teeth or in the buccal cavity.

### DESCRIPTION OF THE DRAWINGS

FIG. 1 depicts the anatomy of a healthy and sensitive tooth

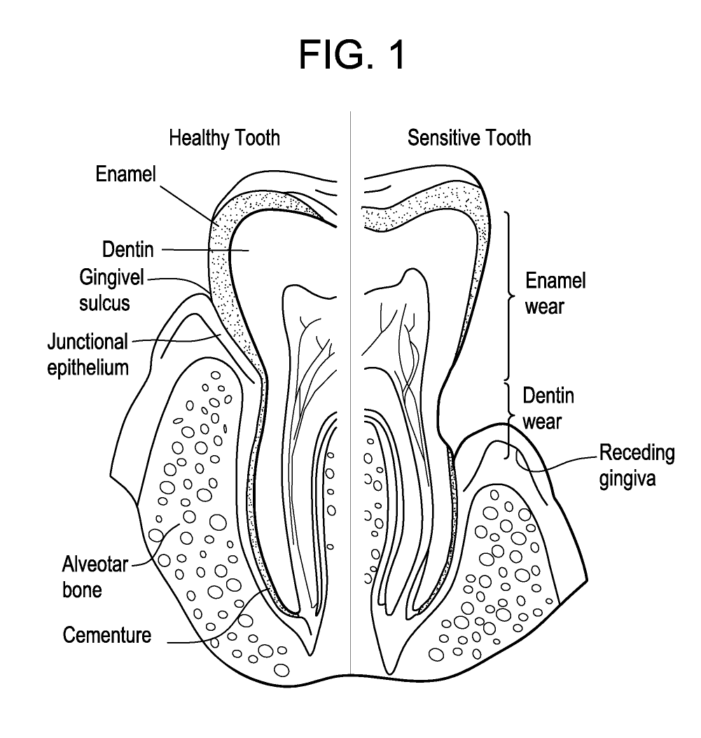

FIG. 2 depicts a micro anatomy of exposed dentin

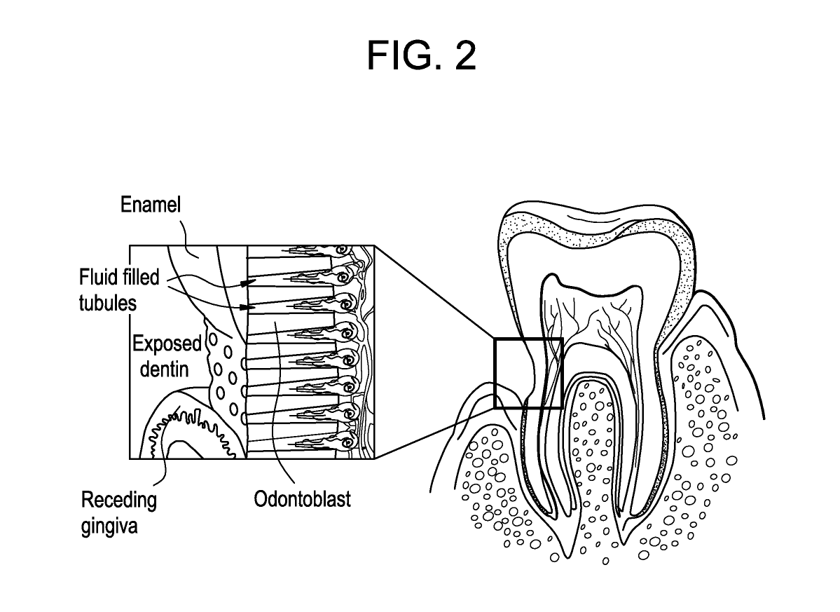

FIG. 3 depicts a specimen of dentin disks cut from human third molars

FIG. 4 depicts an arrangement of dentin disk in Pashley's chamber

FIG. 5 depicts a simple diagram of Pashley's chamber apparatus

FIG. 6 depicts a dentin permeability extinction studies with inventive polymer and commercial polymer.

### DETAILED DESCRIPTION OF THE INVENTION

While applicants do not wish to be bound by the theories, it is believed the inventive compositions provide effective copolymers acting as tooth barriers, and in this way provide improved and effective treatment for sensitive teeth.

The term “tooth sensitivity” refers to painful and irritating condition of tooth exposed to external stimuli such as heat, cold, chemicals, physical and mechanical pressure such as brushing, and flossing causing inward or outward displacement of fluid in the dentinal tubule activating the nerve endings at the pulp/dentin interface.

The term “dentin flow” refers to flow of plasma-like biological fluid (also called dentinal fluid) from the dentin tubules to outer exposed surface of dentin. Dentin flow or permeability is measured in micro liters per minute by in-vitro test method developed by D. H. Pashley & others. Percent flow reduction by treatment can be determined by the following formula:

% Flow reduction=(base line flow−treatment flow)/baseline flow×100

The present inventive synergistic mixture of polymers in the oral care composition results in at least 60% average reduction in dentin flow.

Dentin is the calcified tissue and along with enamel, cementum and pulp form major components of teeth. Dentin consists of microscopic channels, called dentinal tubules, which radiate outward through the dentin from the pulp to the exterior cementum or enamel border. These tubules contain fluid and cellular structures. If enamel wears away, dentin gets exposed to external surface. Changes in the flow of the plasma-like biological fluid present in the dentinal tubules can trigger mechanoreceptors present on nerves located at the pulpal aspect thereby eliciting a pain response. Such condition is termed as teeth sensitivity. This hydrodynamic flow can be increased by cold, air pressure, drying, sugar, sour (dehydrating chemicals), physical pressures are typical triggers for teeth sensitivity. Hence prevention of fluid flow through human dentin is used as a measure to evaluate the tubule occlusion effects of oral care preparations. Specific preparations and their active ingredients that provide greater tubule occlusion should provide a greater potential for reducing teeth hypersensitivity. Polymeric combinations of alkyl vinyl ether and maleic anhydride result in decrease of hydrodynamic flow of fluid into human dentin with improved property of tooth anti-sensitivity. These polymers reduce the dentin flow and thereby act as barrier polymers or tubule blocking agents.

The present invention meets the consumer's needs by providing extinction of sensitivity effect or long lasting effect of desensitivity. It has been surprisingly discovered that synergistic mixtures of polymers having alkyl vinyl ether and maleic acid or anhydride deliver the aforementioned superior results.

The oral care composition described herein comprises a synergistic mixture of

component (A)—a copolymer having at least one repeating unit of alkyl vinyl ether functionality and a monomer having at least one repeating unit of ethylenically unsaturated dicarboxylic acid or anhydride functionality; and
component (B)—a homopolymer having a repeating unit of alkyl vinyl ether functionality.

Non-limiting examples for the repeating unit having alkyl ether functionality can be selected from C₁ to C₂₂ alkyl vinyl ethers including methyl vinyl ether, ethyl vinyl ether, iso-propyl vinyl ether, n-propyl vinyl ether, n-butyl vinyl ether, iso-butyl vinyl ether, tert-butyl vinyl ether, 2-ethylhexyl vinyl ether, n-octyl vinyl ether, n-dodecyl vinyl ether, hexadecyl vinyl ether, octadecyl vinyl ether, isopropenyl methyl ether, 1-butyl vinyl ether, hydroxybutyl vinyl ether, 1,4-cyclohexane-dimethanol monovinyl ether, methoxyethyl vinyl ether, methoxyethoxyethyl vinyl ether, or monomethyl ether of triethylene glycol vinyl ether.

Non-limiting examples for the repeating unit (B) having an ethylenically unsaturated carboxylic acid or anhydride functionality are: maleic acid, maleic anhydride, citraconic acid, citraconic anhydride, itaconic acid, itaconic anhydride, ethylmaleic acid, ethylmaleic anhydride, dimethyl maleic acid, dimethyl maleic anhydride, chloromaleic acid or chloromaleic anhydride.

Accordingly, the present invention provides oral care composition comprising a synergistic mixture of polymers having component (A) in an amount of about 0.1 to about 50% (w/w) and component (B) in an amount of about 0.1 to about 50% (w/w) of the total oral care composition.

Typically, the synergistic mixture of polymers comprises components (A) and (B) present in weight ratio of A to B from 2 to 1 to about 10 to 1, preferably 3 to 1 to 5 to 1. Particularly, good results have been found when (A) to (B) is present in a weight ratio of about 4:1.

In another preferred embodiment, the polymers used in the oral care composition may be selected from methyl vinyl ether/maleic anhydride (or maleic acid) copolymer and methyl vinyl ether homopolymer;

In another preferred embodiment, the present invention provides a total polymer content comprising about 0.1% (w/w) to about 10% (w/w) of the oral care composition, preferably 0.2% to 5% and most preferably 0.3% to 3%.

The synergistic mixture of polymers in the oral care composition has component A with a molecular weight of about 100,000 to 10,000,000 daltons and component B with a molecular weight of about 2,000 to 1,000,000 daltons. Component A preferably has a molecular weight of 500,000-4,000,000, more preferably 800,000-2,000,000. Component B preferably has a molecular weight of 5,000 to 500,000, more preferably 10,000 to 200,000.

In another embodiment, the composition of the present invention includes oral care additives such as antibacterial agents, anti-inflammatory agents, humectants, surfactants, detergents, anticaries agents, whitening agents, anti-cavity or enamel repair agents, breath freshening agents, sweeteners, flavor oils, foaming agents, solubilizing agents, abrasives, tartar control or anti-stain ingredients, anti-erosion ingredients, fluoride sources, gums, stabilizers, opacifying agents, pH buffering agents, chelating agents, colorants, enzymes, sensate ingredients, and/or their combinations.

Examples of antibacterial agents include, but are not limited to, zinc salts, stannous salts, cetyl pyridinium chloride, chlorhexidine, triclosan, triclosan monophosphate, and flavor oils. Triclosan is the preferred anti-bacterial agent of the invention, preferably utilized in amounts of about 0.03% to about 0.6% by weight of the total oral care composition.

Anti-caries agents such as stannous fluoride, sodium and potassium fluoride, sodium and potassium monofluorophosphate, preferably sodium fluoride, may be incorporated into the oral care compositions in amounts well known in the art. The fluoride compositions prevent formation of cavities and strengthen the enamel surface.

Preservatives which may be used in the oral care formulations of the invention include those conventionally employed in the art, such as benzoic acid and sodium benzoate; the parabens; sorbic acid and sorbates; propionic acid and propionates; acetic acid and acetates; nitrates and nitrites; sulfur dioxide and sulfites; antibiotics; diethyl pyrocarbonate; epoxides; hydrogen peroxide; and phosphates. The parabens include the methyl, ethyl, propyl, and butyl esters of parahydroxybenzoic acid.

Suitable solvents for the present composition include water, edible polyhydric alcohols such as glycerin, diglycerin, triglycerin, sorbitol, xylitol, butylenes glycol, erythritol, polyethylene glycol, propylene glycol, and combinations thereof.

Thickeners useful in the oral care composition of the present invention include natural and synthetic gums, hydrocolloids, cellulose derivates, and silicates. These include hydrated silica, colloidal silica, abrasive silica, sodium carboxymethyl cellulose, polyvinyl pyrrolidone, methyl cellulose, hydroxypropyl cellulose, Carageenan and mixtures thereof as known in the art. Preferably the thickener is abrasive silica, thickening silica.

Humectants include glycerin, propylene glycol, butylenes glycol, sodium hyaluronate, hyaluronic acid, caprylyl glycol, cyclomethicone, glycerol, Sorbitol, lactic acid, urea, algae extract, sucrose, trehalose, hydrolysed soy protein, hydrolysed collagen, betaine and sodium lactate, preferably glycerol. These compounds promote the retention of moisture.

Sweeteners include those well known in the art. The sweetening agent may be selected from a wide range of materials including water-soluble agents, water-soluble artificial sweeteners, and dipeptide based sweeteners, including mixtures thereof. Without being limited to particular sweeteners, representative illustrations encompass:

A. water-soluble sweetening agents such as monosaccharides, disaccharides and polysaccharides such as xylose, ribose, glucose, mannose, galactose, fructose, dextrose, sucrose, sugar, maltose, partially hydrolyzed starch, or corn syrup solids and sugar alcohols such as sorbitol, xylitol, mannitol, maltitol, hydrogenated starch hydrolysate and mixtures thereof.
B. water-soluble artificial sweeteners such as the soluble saccharin salts, i.e., sodium or calcium saccharin salts, cyclamate salts, acesulfam-K, sucralose and the like, and the free acid form of saccharin
C. dipeptide based sweeteners such as L-aspartyl-L-phenylalanine methyl ester and materials described in U.S. Pat. No. 3,491,131, L-D-aspartyl-N-(2,2,4,4-tetramethyl-3-thietanyl-D-alaninamide hydrate) and the like.
In general, the amount of sweetener will vary with the desired amount of sweetener selected for a particular oral care formulation.

Flavoring agents well known to those in the oral care arts may be added to the compositions of the instant invention. These flavoring agents may be chosen from synthetic flavor oils and/or oils derived from plants, leaves, flowers, fruits and so forth, and combinations thereof. Representative flavor oils include: spearmint oil, cinnamon oil, oil of wintergreen (methyl salicylate) and peppermint oils. Also useful are artificial, natural or synthetic fruit flavors such as citrus oil including lemon, orange, grape, lime and grapefruit, and fruit essences including apple, strawberry, cherry, pineapple and so forth. The flavoring agent may be a liquid, spray dried, encapsulated, sorbed on a carrier and mixtures thereof. A preferred flavoring agent is peppermint oil, commercially available from Rose Mitcham. The amount of flavoring agent utilized may vary depending on such factors as flavor type, oral care formulation and strength desired. In general, amounts of about 0.01% to about 5.0% by weight of the total oral care composition are usable, with amounts of about 0.05% to 0.15% being preferred.

Surfactants include alkyl sulfate, alkyl aryl sulfates, fatty alcohol sulfates, higher fatty acid sulfonates such as dodecyl sulfonate, and sodium lauroyl sarcosinate.

The colorants useful in the present invention include pigments such as titanium dioxide, and may also include dyes suitable for food, drug and cosmetic applications. These colorants are known as F.D. & C dyes. The materials acceptable for the foregoing spectrum of use are preferably water-soluble. Illustrative examples include indigo dye, known as F.D. & C. blue No. 2, which is the disodium salt of 5,5′-indigotin-di-sulfonic acid. Similarly, the dye known as F.D. & C. Green No. 1, comprises a triphenylmethane dye and is the monosodium salt of the 4-[4-N-ethyl-p-sulfobenzylamino)diphenylmethylene]-[1-N-ethyl-N-p-sulfonium-benzyl)-2,5-cyclohexa-dienimine]. A preferred colorant is F.D. & C. Red No. 3. A full recitation of F.D. & C. and D. & C. colorants and their corresponding chemical structures may be found in the Kirk-Othmer Encyclopedia of Chemical Technology, 3rd Edition, in Volume 6, at pages 561-595. In general, colors when used are employed in amounts of about 0.005% to about 0.5% by weight of the oral care composition.

In another aspect, the present makes use of a known industry standard test method for measuring the hydrodynamic flow through human dentin. The most accepted explanation for tooth sensitivity or dentin hypersensitivity is based on Brannstrom's hydrodynamic work [Reference: Brannstrom, M.; the hydrodynamic theory of dentinal pain: sensation in preparations, caries, and dental crack syndrome; J. Endod. 1986; 12; 453-7]. This theory shows that various stimuli such as cold, heat, pressure, or sweet cause inward or outward displacement of the fluid in the dentinal tubules activating the nerve endings at the pulp/dentin interface, resulting in pain. Decreasing the fluid flow or (dentin permeability) by occlusion of dentin tubules has been recognized as a very effective treatment for tooth sensitivity. Such measurement of dentin flow can be used as a means to evaluate the tubule occlusion effects of oral care preparations. Active ingredients that provide greater tubule occlusion possess greater potential in reducing the tooth sensitivity.

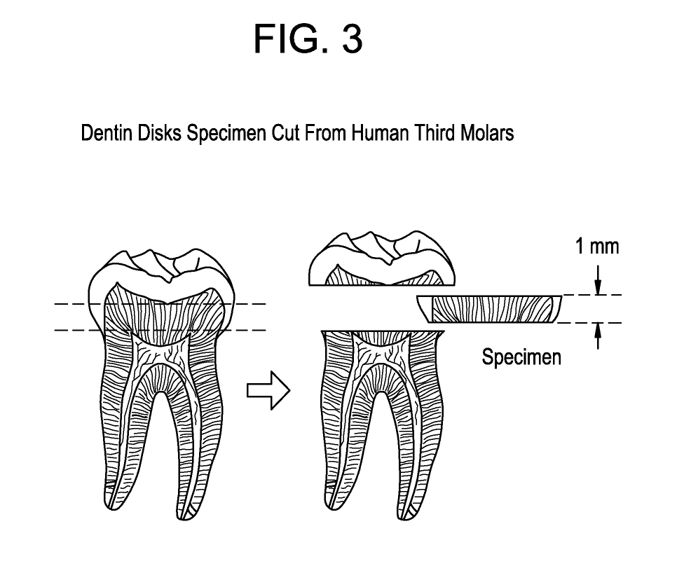

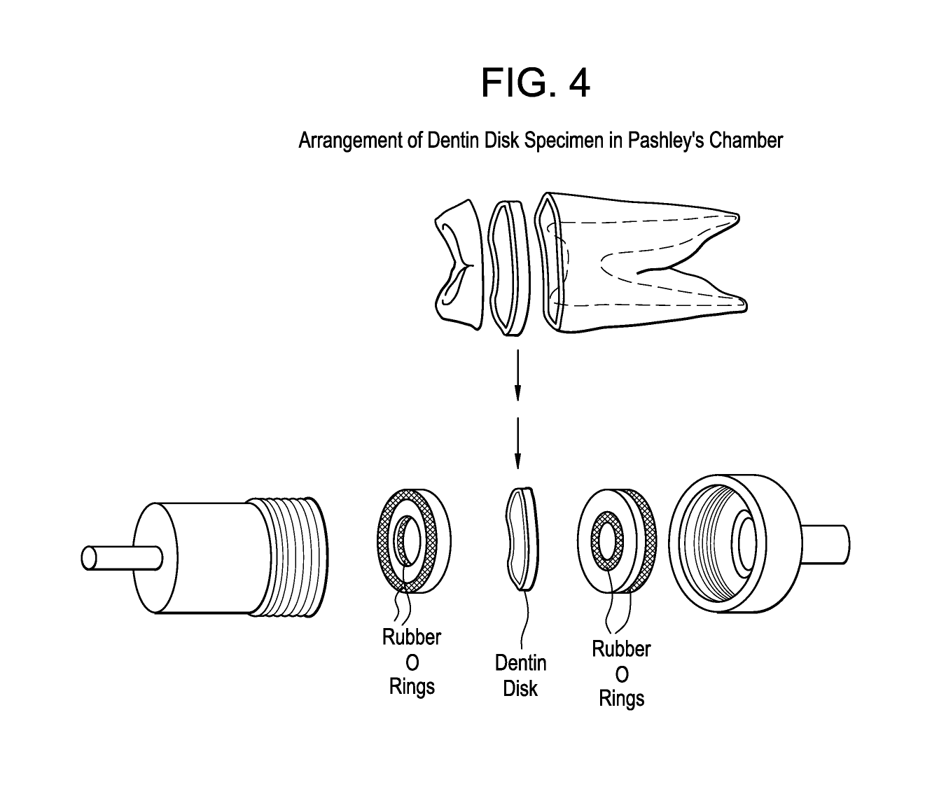

The in-vitro test method has been developed by D. H. Pashley and others [Reference: Outhwaite, William C; Mckenzie, Donald M.; and Pashley, David H.; “A versatile split-chamber device for studying dentin permeability”]. In this method, dentin disks are cut from human third molars and clamped between two pairs of rubber “O” rings inside a chamber to give a constant exposed dentin surface (shown in FIGS. 3 and 4). The system is connected by tubing to a reservoir of water that provides constant water pressure. The hydrodynamic flow through these human dentin disks is measured as a means to evaluate the dentin tubule occlusion effects of oral care preparations. The percent flow reduction between untreated and treated dentin disks is an in-vitro quantification of the anti-sensitivity effect.

### Flow Determination

Dentin flow or permeability is measured in micro liters per minute. Percent flow reduction by treatment can be determined by the following formula:

% Flow reduction=(base line flow−treatment flow)/baseline flow×100

Pashley's in-vitro test method is used for measuring the dentin flow and accordingly present inventive polymers are tested for average reduction in dentin flow. Oral care composition containing the inventive combination of polymers showed an average reduction in dentin flow from about 60-95%, preferably 70-90% and more preferably 75-85%.

In another preferred embodiment, the inventive compositions can be incorporated into formulations such as toothpastes, powders, sprays, suspensions, varnishes, sealants, coatings, oral strips, mouth washes, mouth rinses, teeth whitening compositions or boosters, anti-calculus formulations, anti-gingivitis formulations, instant powders, pastilles, oral gels, chewing gums, buccal patches, or lozenges.

In another preferred embodiment, the present invention provides a process for preparing oral care compositions comprising synergistic mixture of polymers and other oral care additives.

The following non-limiting examples illustrate specific embodiments of the present invention. They are not intended to be limiting the scope of the present invention in any way.

### EXAMPLES

### Example 1

### Preparation of Methyl Vinyl Ether/Maleic Anhydride Copolymer

The inventive oral care composition can be prepared using polymers known in the art. In particular, methyl vinyl ether/maleic anhydride copolymer can be used for the inventive compositions. Such copolymers are manufactured by Ashland and available under the trade name Gantrez S-97.

### Example 2

### Method for Preparing Tooth Paste Formulation

A tooth paste formulation was prepared using copolymer along with oral care additives as disclosed in formula 1. For comparison studies, composition without synergistic mixture was prepared as disclosed in formula 2 which acts as control sample.

TABLE 1

% (w/w)

| Ingredient | Formula 1 | Formula 2 |
|---|---|---|
| Triclosan | 0.300 | 0.300 |
| Sodium fluoride | 0.243 | 0.243 |
| Glycerin, USP 99.5% | 20.000 | 20.000 |
| Sorbitol solution (70%) USP | 20.000 | 20.000 |
| Abrasive silica | 20.000 | 20.000 |
| Thickening silica | 2.000 | 2.000 |
| SLS, USP/NF | 1.500 | 1.500 |
| Sodium hydroxide 50% | 1.200 | 1.200 |
| solution | | |
| Peppermint Oil (SAFC, | 1.000 | 1.000 |
| FCC) | | |
| Sodium CMC 7MF | 0.800 | 0.800 |
| Titanium dioxide | 0.500 | 0.500 |
| Carageenan | 0.300 | 0.300 |
| Sodium saccharin | 0.300 | 0.300 |
| Poly (methyl vinyl ether/ | 2.000 | None |
| Maleic anhydride) | | |
| Poly (methyl vinyl ether) | 0.500 | |
| Water | q.s. to 100 | q.s. to 100 |

### Example 3

### Test Method to Determine the Dentin Tubule Occlusion

Oral Care composition as prepared in example 2 was taken and tested using an industry standard in-vitro test method to measure the hydrodynamic flow through human dentin. Measurement of dentin flow helps us to evaluate the tubule occlusion effects of oral care preparations. Active ingredients that provide greater tubule occlusion possess greater potential in reducing the tooth sensitivity. The Pashley's in-vitro test procedure is provided below:

### Dentin Disks

Disinfected human third molars were taken and sectioned with precision diamond blade parallel to occlusion surface at the point just cervical to dentinal-enamel junction and another point coronal to the pulp horns. Sizes of the disks were cut with precision to fit into the Pashley's chamber. Disks were etched in >5 ml of 50% W/V citric acid for three (3) minutes to remove the “smear layer” from the microtubules and were held in freshly prepared PBS solution for at least 30 minutes before measurement.

Pashley's Chamber and Apparatus

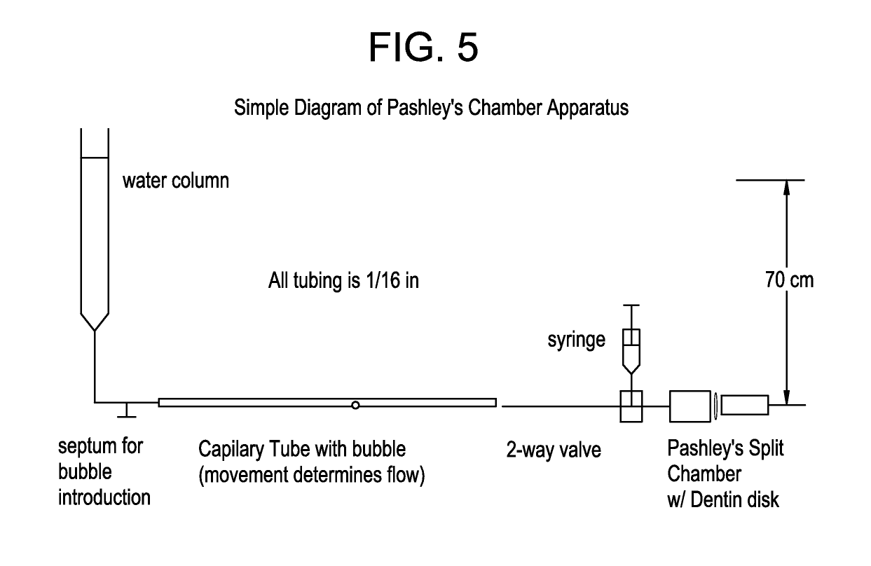

FIG. 5 represent simple diagram of Pashley's Chamber Apparatus. Untreated disks were individually identified by numbering on their edge in pencil. Orientation of each disk in the chamber (# side up or down) in the pre-treatment phase was recorded and the same orientation was used in the post-treatment phase. Each numbered disk was tested in the same chamber in both the pre-treatment and post-treatment phases.

Disk Treatment

Disks were treated in the following different combinations:

i. disks were treated with T/P according to label directions or

ii. tooth paste aqueous phase (TA/P) was used undiluted or

iii. 2.5 g T/P was mixed with 4.0 g D.I. water

Tooth paste or tooth paste aqueous phase was placed in a test tube and a disk was immersed with slight shaking at 37° C. for 30 minutes. Disks were removed from T/P and rinsed by dipping three times in a large excess (>2 liters) of D.I. water. Disks were placed in Pashley's chamber for measurement after treatment.

Measurement in Pashley's Chamber and Apparatus

### Initial Start-Up

The Pashley's Chamber was filled with 5% ethanol/water (V/V) and allowed to rest in the off position for at least a day before use. Directly before use, the ethanol/water was drained to the bottom of the pipette (just visible) and filled with D.I. water to give the appropriate head pressure (usually 70 cm). The valve was turned to open the syringe fitting and water is drawn through the system by pulling the large syringe. A bubble was introduced through the septa with a 100 micro liter syringe, pushing the needle to the bottom of the fitting and injecting about 40 micro liters of air. The bubble was drawn into the beginning of the capillary tube with a 3 ml syringe. The reservoir was refilled to the correct head pressure and the position of the bubble is measured or marked. The position of the bubble was measured from the “zero time” position and recorded.

After initial start-up was complete and the bubble was confirmed to be stationary for 15 minutes, testing was started. Dentin disks were placed individually in Pashley's chamber between two appropriately sized “O” rings and the split chamber sections were screwed together until finger tight. Chamber was placed back in its holder. The exact location of the capillary bubble was measured and allowed water to flow into the chamber through the open valve. The bubble's location or movement was measured at an interval of 15 minute up to 90 minutes.

Flow Determination

Dentin flow or dentin permeability was determined in micro liters per minute. Percent flow reduction by treatment was determined by the following formula:

% Flow Reduction=(Baseline flow−Treatment flow)/Baseline flow×100.

Accordingly, commercially available polymers were formulated into toothpaste aqueous phase (TA/P) and evaluated for dentin tubule occlusion efficacy by Pashley's chamber testing method.

Table 1 lists the compositions of inventive polymers. All dentin disks have different and unique base, or untreated, flow rates and this is reflected in the treated flow rates. To normalize these natural variations in dentin, the average percent reduction in flow rate is calculated. The average percent reduction in flow is the average of the individual averages for all three dentin disks (both pre and post treatment). Each average percent reduction then, is a result of 36 individual flow rate measurements.

Dentin flow results for each dentin disk in μl/min from six (6) measurements at 15 minute intervals over 90 minutes total were measured. Average results of all three disks over the entire 90 minute flow period are provided in Table 2 below:

TABLE 2

| | | Avg Post- | Avg % | Avg % |
|---|---|---|---|---|
| Polymers used in | Avg Pre- | Treat | Immediate | Extinction |
| T/AP Treatments to | Treat | Extinction | Flow | Flow |
| Dentin Disks | Flow | Flow (20 hrs) | Reduction | Reduction |
| 2% Gantrez S-97 | 1.85 | 0.86 | 95.34 | 54.40 |
| Control w/ 0.5% | | | | |
| PMVE | | | | |
| 4% Chevron PA-18 | 2.33 | 1.18 | 65.53 | 36.77 |
| Gantrez Control | 1.97 | — | 62.70 | — |

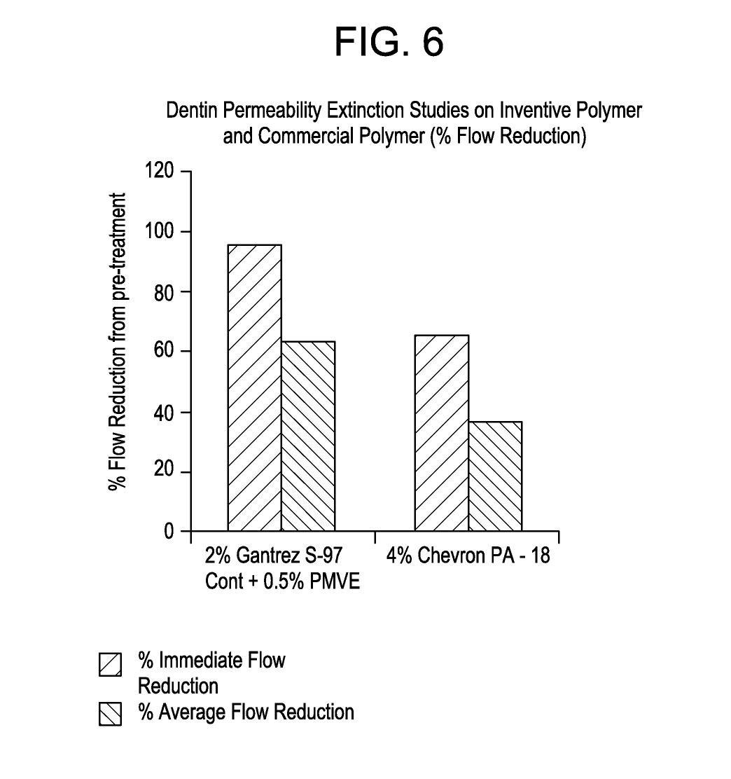

FIG. 6 provides graphical representation of dentin flow or permeability extinction studies on inventive combination of polymers and compared with commercial polymers. As seen in Table 2, the formulation with synergistic mixture of polymers methyl vinyl ether/maleic anhydride copolymer and methyl vinyl ether homopolymer showed 95.34% of average immediate reduction in dentin flow, while the commercial polymer 4% Chevron PA-18 showed an average immediate flow reduction of 65.53%, thus making the inventive composition as a highly effective tooth paste formulation for treating tooth sensitivity. In similar experiments where the weight ratio of (A) to (B) of Gantrez S-97 t PMVE was 2 to 1, a good average % reduction in flow rate was observed.

While the present invention has been illustrated by the description of embodiments and examples thereof, and while the embodiments have been described in considerable detail, they are not intended to restrict or in any way limit the scope of the appended claims to such detail. Accordingly, departures may be made from such details without departing from the scope of the general inventive concept.

We claim:

1. An oral care composition for treating tooth sensitivity comprising a synergistic mixture of:
component (A)—a copolymer having at least one repeating unit of alkyl vinyl ether functionality and a monomer having at least one repeating unit of ethylenically unsaturated dicarboxylic acid or anhydride functionality present in an amount of about 0.1 to about 50% (w/w) of the total mixture; and
component (B)—a homo polymer having repeating unit of alkyl vinyl ether functionality present in an amount of about 0.1 to about 50% (w/w) of the total mixture;
wherein the composition exhibits at least 60% average reduction in dentin flow.

2. The oral care composition according to claim 1, wherein said repeating unit of alkylvinyl ether functionality is selected from the group consisting of methyl vinyl ether; ethyl vinyl ether; iso-propyl vinyl ether; n-propyl vinyl ether; n-butyl vinyl ether; iso-butyl vinyl ether; tert-butyl vinyl ether; 2-ethylhexyl vinyl ether; n-octyl vinyl ether; n-dodecyl vinyl ether; octadecyl vinyl ether; isopropenyl methyl ether; 1-butyl vinyl ether; hydroxybutyl vinyl ether; 1,4-cyclohexane-dimethanol monovinyl ether; methoxyethyl vinyl ether; methoxyethoxyethyl vinyl ether; the monomethyl ether of triethylene glycol vinyl ether.

3. The oral care composition according to claim 1, wherein said repeating unit of ethylenically unsaturated carboxylic acid or anhydride functionality consists of maleic acid, maleic anhydride, citraconic acid, citraconic anhydride, itaconic acid, itaconic anhydride, ethylmaleic acid, ethylmaleic anhydride, dimethyl maleic acid, dimethyl maleic anhydride, chloromaleic acid or chloromaleic anhydride.

4. The oral care composition according to claim 1, wherein said composition is a synergistic mixture of methyl vinyl ether/maleic anhydride copolymer and homopolymer of methyl vinyl ether.

5. The oral care composition according to claim 1, wherein the components (A) and (B) are present in a weight ratio of A:B from 2:1 to 10:1.

6. The oral care composition according to claim 1, wherein said synergistic mixture is present in an amount of about 0.1% (w/w) to about 10% (w/w) of the total oral care composition.

7. The oral care composition according to claim 1, wherein said synergistic mixture has a component (A) with molecular weight of 100,000 to 10,000,000 Daltons and component (B) with molecular weight of 2,000 to 1,000,000 Daltons.

8. The method of treating tooth sensitivity comprising applying to a tooth and/or a buccal cavity the oral care composition of claim 1.

9. The oral care composition according to claim 1, further comprising additives selected from the group consisting of antibacterial agents, anti-inflammatory agents, humectants, surfactants, detergents, anticaries agents, whitening agents, anti-cavity or enamel repair agents, breath freshening agents, sweeteners, flavor oils, foaming agents, abrasives, tartar control or anti-stain ingredients, anti-erosion ingredients, fluoride sources, gums, stabilizers, colorants, enzymes, sensate ingredients, and/or their combinations.

10. The oral care composition according to claim 1, wherein said composition is incorporated into a toothpaste, a tooth powder, a mouth wash, a mouth rinse, a teeth whitening composition or booster, an anti-calculus formulation, an anti-gingivitis formulation, an oral gel a chewing gum, or a lozenge.

11. Method for alleviating the symptoms of tooth sensitivity, acid erosion, caries or stains comprising topically applying the oral care composition of claim 1 on teeth or in the buccal cavity.

12. An oral care composition for treating tooth sensitivity comprising a synergistic mixture of:
a) about 2% w/w of methyl vinyl ether/maleic anhydride copolymer;
b) about 0.5% w/w of poly (methyl vinyl ether); and
c) about 97.5% of w/w oral care additives, or actives or ingredients.

13. The composition according to claim 12, wherein said composition exhibits an average immediate reduction of at least 80% in dentin flow.

14. The oral care composition according to claim 12, wherein said composition is incorporated into a toothpaste, a tooth powder, a mouth wash, a mouth rinse, a teeth whitening composition or booster, an anti-calculus formulation, an anti-gingivitis formulation, an oral gel a chewing gum, or a lozenge.

15. Method for alleviating the symptoms of tooth sensitivity, acid erosion, caries or stains comprising topically applying the oral care composition of claim 12 on teeth or in the buccal cavity.
